(19) United States
(12) Patent Application Publication
(10) Pub. No.: US 2020/0114323 A1
(43) Pub. Date: Apr. 16, 2020

(54) SYSTEMS AND METHODS FOR TREATING AND CONDITIONING SMALL VOLUME LIQUID SAMPLES

(71) Applicant: Feistel Holding Corp., Laguna Beach, CA (US)

(72) Inventor: Christopher Feistel, Laguna Beach, CA (US)

(21) Appl. No.: 16/596,944

(22) Filed: Oct. 9, 2019

Related U.S. Application Data
(60) Provisional application No. 62/745,256, filed on Oct. 12, 2018.

(51) Int. Cl. B01F 13/00; G01N 33/569

(52) U.S. Cl. CPC B01F 13/0006; B01F 2215/0037; G01N 33/56966

(57) ABSTRACT

Systems and methods are for creating liquid toroidal mixing patterns within microliter volumes of liquids are contemplated. An electrode geometry comprising peripheral electrodes defining an annular configuration and interior electrodes are contemplated, to which an alternating current may be applied. Via control of the frequency, waveform, amplitude and bias of the current, liquids and the components of those liquids may be mixed in a toroidal motion. This same electrode geometry may also be used to accomplish the deposition of targeted materials onto the surface of the peripheral or interior electrode by further manipulating the frequency, amplitude, and bias of the waveform of the applied current. Methods of applying such techniques are also contemplated in the context of blood typing assays, latex agglutination assays, micro array assays, transfection, transduction, and tissue engineering methods, and it may be seen that such techniques may result in substantial benefits.

### CROSS-REFERENCE TO RELATED APPLICATIONS

This application relates to and claims the benefit of U.S. Provisional Application No. 62/745,256 filed Oct. 12, 2018 entitled “SYSTEMS AND METHODS FOR TREATING AND CONDITIONING SMALL VOLUME LIQUID SAMPLES,” the entire disclosure of which is hereby wholly incorporated by reference.

### STATEMENT RE: FEDERALLY SPONSORED RESEARCH/DEVELOPMENT

Not Applicable

### BACKGROUND

### 1. Technical Field

The present disclosure relates generally to systems and methods for mixing small volumes of liquids, and more particularly to systems and methods for mixing small volumes of liquid using novel electrophoretic processes.

### 2. Related Art

Those skilled in the arts of molecular biology, bio-engineering, biomedical engineering, bio-manufacturing, bio-technology, molecular engineering, medical diagnostics, blood banking and others recognize the need, and the benefits, of using microliter volumes of liquid for a great variety of important commercial and academic applications. However, the process of mixing liquids using mechanical processes such as stirring, shaking, bubbling, rocking, and boiling (convection) become ineffective or infeasible when used in microliter liquid volumes, particularly when volumes are less than 25 ul.

In recent years, the field of microfluidics has developed a variety of prior art systems and methods for liquid mixing and separation processes. For example, it is known that liquid mixing and separation processes may be performed using dielectrophoresis and/or electro osmosis principles in combination with linear and non-linear microchannels (straight , branched, serpentine, spiral) or even tortured paths (incorporating physical obstacles such as micro pillars or micro herring bone protrusions into the microfluidic pathway) that have an initiation point and end point, said initiation point and end point having different physical coordinates. One example of such a microchannel-based microfluidic mixing technique may be found in the recently filed U.S. Patent Application Publication 201810093232 A1, entitled “Bifurcating Mixers and Methods of their Use and Manufacture.” That application that teaches the utility of moving microvolumes of liquid through micro channels configured as a series of angled toroidal mixers to accomplish mixing.

Many microfluidics-based devices today are popularly known as “labs-on-a-chip.” Labs-on-a-chip generally rely on active handling of fluid flow through complex networks of channels or porous media. However, the broad adoption of labs-on-a-chip along with other microfluidic methods and devices for large scale commercial applications, such as medical diagnostics, is challenged by complex high cost fabrication processes, low production yields, field performance throughput that is too slow to meet market demand, and unreliable performance. All of these challenges directly result from this reliance on complex networks of microchannels, microchambers, and liquid coupled electronics.

Thus, it may be seen there exists a need in the art for novel microfluidic systems and methods that remedy these and other deficiencies.

### BRIEF SUMMARY

To solve these and other problems, systems and methods are contemplated for creating liquid toroidal mixing patterns within microliter volumes (droplets) of liquids. According to various embodiments of such systems, electrode geometries comprising arrangements of peripheral and interior electrodes are contemplated, to which an alternating current may be applied. Via control of the frequency, waveform, amplitude and bias of the applied current, liquids and the components of those liquids may be mixed in a toroidal motion centered around the interior electrode(s). It is further contemplated that these same electrode geometries may also be used, in addition to toroidal mixing, to accomplish the deposition of targeted materials onto the surface of the peripheral or interior electrode(s) by further manipulating the frequency, amplitude, and bias of the waveform of the applied current. Thus, it may be seen that the presently contemplated disclosure may permit an effective mixing of liquids mixing in microliter volumes without the use of mechanical and other active devices such as stirrers, fluid pumps, acoustic energy generators (e.g., ultrasonic mixers), or heat pumps, and that further manipulations of the liquids or the constituents thereof may be accomplished without requiring transfer of the liquid to a separate physical location.

According to an exemplary embodiment of the present disclosure, a system for inducing toroidal mixing in a volume of liquid is contemplated, with the system comprising a substrate and an electrode geometry at the surface of the substrate, the electrode geometry comprising one or more peripheral electrodes, the one or more peripheral electrodes together defining at least a partial annulus, and one or more interior electrodes, the one or more interior electrodes being disposed interior to the at least partial annulus defined by the one or more peripheral electrodes, wherein when a volume of liquid is disposed in electrical contact with at least one of the one or more peripheral electrodes and at least one of the one or more interior electrodes, and when at least a first electrical current is applied to the electrode geometry, the system is operative to induce toroidal mixing in the volume of liquid.

The at least partial annulus defined by the one or more peripheral electrodes may be characterized as circular, elliptical, ovoid, polygonal, or combinations thereof. The one or more interior electrodes may be configured to be substantially concentric with the at least partial annulus defined by the one or more peripheral electrodes, and the one or more interior electrodes may be characterized as: circular, semicircular, elliptical, semi-elliptical, ovoid, semi-ovoid, annular, semi-annular, polygonal, star-shaped, or combinations thereof

The electrode geometry may be at least partially disposed within a well for at least partially containing the volume of liquid. The system may further comprise a membrane at least partially enclosing at least a portion of the well, the membrane being configured to be substantially permeable to at least one component of the volume of liquid and substantially impermeable to a second component of the volume of liquid. According to specific embodiments, the volume of liquid may comprise a plurality of cells, and the membrane may be substantially impermeable to the plurality of cells while remaining permeable to one or more other components of the volume of liquid.

The system may further comprise a signal generator for generating the first electrical current. The first electrical current may be an alternating current, and may be characterized as a continuous waveform, a pulsed (also known as burst) waveform, a sinusoidal waveform, a non-sinusoidal waveform, or combinations thereof.

The substrate may comprise a printed circuit board. The system may also comprise an array having a plurality of electrode geometries at the surface of one or more substrates.

The system may be further operative to induce dielectrophoretic separation among components of the volume of liquid via application of a second electrical current to the electrode geometry. The dielectrophoretic separation is operative to cause at least one component of the volume of liquid to aggregate at a dielectrophoretic aggregation site. The second electrical current may be one or more of: an alternating current, a direct current, a variable current.

Various methods are contemplated for inducing toroidal mixing in a volume of liquid. According to an exemplary embodiment, such a method may comprise the steps of providing an electrode geometry at the surface of a substrate, the electrode geometry comprising one or more peripheral electrodes, the one or more peripheral electrodes together defining at least a partial annulus, and one or more interior electrodes, the one or more interior electrodes being disposed interior to the at least partial annulus defined by the one or more peripheral electrodes, disposing the volume of liquid in electrical contact with at least one of the one or more peripheral electrodes and at least one of the one or more interior electrodes, and applying a first electrical current to the electrode geometry, the first electrical current being an alternating current. According to a more particular embodiment of such a method, a volume of liquid is utilized which is between 100 nL and 5 mL.

According to a more specialized embodiment, a method of performing a blood-type assay is additionally contemplated. Specifically, such a method may comprise the steps of providing an electrode geometry at the surface of a substrate, the electrode geometry comprising one or more peripheral electrodes, the one or more peripheral electrodes together defining at least a partial annulus, and one or more interior electrodes, the one or more interior electrodes being disposed interior to the at least partial annulus defined by the one or more peripheral electrodes, disposing a volume of liquid comprising erythrocytes from a first individual and plasma or serum from another individual in electrical contact with at least one of the one or more peripheral electrodes and at least one of the one or more interior electrodes, inducing toroidal mixing in the volume of liquid via applying a first electrical current to the electrode geometry, the first electrical current being an alternating current, aggregating the erythrocytes at a dielectrophoretic aggregation site by dielectrophoretic separation via applying a second electrical current to the electrode geometry, displacing the plasma or serum from the dielectrophoretic aggregation site, and exposing the dielectrophoretic aggregation site to a solution comprising anti-human IgG.

According to further refinements of the above contemplated method of performing a blood-type assay, it is contemplated that the dielectrophoretic aggregation site may be chosen from: at least one of the one or more peripheral electrodes, at least one of the one or more interior electrodes, another electrode, a region of an electric field, or combinations thereof. It is also contemplated that the step of displacing the plasma or serum may be performed via application of a wash solution. It is further contemplated that the above contemplated method of performing a blood-type assay may further include a step of detecting the relative presence or relative absence of cross-linked erythrocytes.

### BRIEF DESCRIPTION OF THE DRAWINGS

These and other features and advantages of the various embodiments disclosed herein are better understood with respect to the following descriptions and drawings, in which:

FIG. 1A is a top view of an embodiment of an electrode geometry according to the present disclosure having a circular interior electrode configuration;

FIG. 1B is a top view of an embodiment of an electrode geometry according to the present disclosure having a hexagonal interior electrode configuration;

FIG. 1C is a top view of an embodiment of an electrode geometry according to the present disclosure having an annular interior electrode configuration;

FIG. 1D is a top view of an embodiment of an electrode geometry according to the present disclosure having a semi-annular interior electrode configuration;

FIG. 1E is a top view of an embodiment of an electrode geometry according to the present disclosure having a semicircular interior electrode configuration;

FIG. 2A is a top view of a volume of liquid disposed in electrical contact with an exemplary embodiment of an electrode geometry;

FIG. 2B is a perspective view of a volume of liquid disposed in electrical contact with an exemplary embodiment of an electrode geometry;

FIG. 2C is a side view of a volume of liquid disposed in electrical contact with an exemplary embodiment of an electrode geometry;

FIG. 3 is a perspective view of an exemplary embodiment of an electrode geometry showing electromagnetic forces that induce toroidal mixing;

FIG. 4A is a schematic illustrating an embodiment of toroidal mixing resulting from a continuous waveform;

FIG. 4B is a schematic illustrating an embodiment of toroidal mixing resulting from a pulsed waveform;

FIG. 5A is a schematic illustrating an exemplary embodiment of an array of electrode geometries on the surface of a substrate;

FIG. 5B is a schematic illustrating an exemplary embodiment of a circuit layer underneath the surface of the substrate of FIG. 5A;

FIG. 6 is a perspective cutaway view of one embodiment of the present disclosure wherein the electrode geometry is positioned at the base of a well, the volume of liquid comprises a plurality of cells, and a membrane is positioned within the well that is substantially impermeable to the plurality of cells; and

FIG. 7 is a schematic illustrating an exemplary embodiment of an electrode geometry showing electromagnetic forces that induce dielectrophoretic separation and aggregation at a dielectrophoretic aggregation site.

Common reference numerals are used throughout the drawings and the detailed description to indicate the same elements.

### DETAILED DESCRIPTION

According to exemplary aspects of the present disclosure, systems and methods are contemplated for inducing toroidal mixing within microliter volumes (droplets) of liquids. In particular, a system is contemplated which comprises an electrode geometry at the surface of a substrate, with the electrode geometry being formed of one or more peripheral electrodes together defining at least a partial annulus and one or more interior electrodes being positioned interior to the at least partially defined annulus. When a volume of liquid is disposed in electrical contact with at least one of the peripheral electrodes and at least one of the interior electrodes, and when a first electrical current having certain characteristics is applied to the electrode geometry, the system is operative to induce toroidal mixing. Further, the same electrode geometry may be utilized, when a second electric current having other characteristics is applied, to induce dielectrophoretic separation among components of the volume of the liquid, which may result in aggregation of at least one component at a dielectrophoretic aggregation site, such as one of the electrodes. It may thus be seen that through manipulation of the applied current, both mixing and dielectrophoretic separation may be carried out upon a single liquid sample, which may be as small as a microliter volume droplet, without requiring the liquid sample to be transported, thus achieving substantial gains in efficiency and simplicity. Through application of these herein described techniques, it may be seen that certain novel methods of performing certain liquid conditioning schemes, such as immunoassays, molecular assays, blood typing, and cell transections and transductions are able to be performed in fashions that are substantially simplified, speedier, and less expensive.

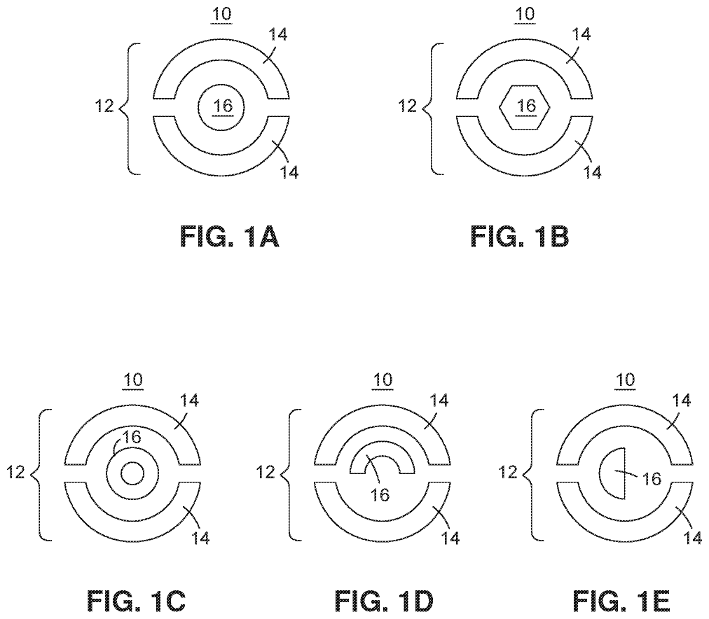

Turning now to FIG. 1, various embodiments of systems 10 for inducing toroidal mixing in a volume of liquid are illustrated. The system 10 for inducing toroidal mixing in a volume of liquid 20 may comprise an electrode geometry 12 formed from one or more peripheral electrodes 14 and one or more interior electrodes 16, with the electrode geometry 12 being positioned at the surface of a substrate 18 (not pictured).

According to the various illustrated embodiments shown in FIG. 1, the electrode geometry may comprise one or more peripheral electrodes 14 together defining at least a partial annulus. An annulus is a ring shape, and the one or more peripheral electrodes 14 may partially define the general shape of a ring, though they do not necessarily, and indeed, in most embodiments are not contemplated as forming a complete annulus, but rather merely partially define a general annular shape. In each of the exemplary embodiments illustrated in FIGS. 1A through 1E, there are two peripheral electrodes 14 which are configured such that each peripheral electrode 14 forms a partial hemisphere of a rounded, circular annulus, and together the two peripheral electrodes 14 define a partial annulus, with a gap in between the two partial hemispheres. However, it may be seen that in other embodiments, the peripheral electrodes may be configured in other configurations. For example, but without limitations, the peripheral electrodes 14 may form an essentially complete annulus (which may be a complete annulus where there is a single electrode, or may form a substantially less complete annulus than illustrated in the exemplary embodiment of FIG. 1, such as if there are more than two electrodes which form the annulus, with correspondingly more gaps between the electrodes. In certain contemplated embodiments in which there is only a single peripheral electrode 14, that peripheral electrode 14 may only have a single gap at one point in the annulus, or may have no gaps and may define a complete annulus.

Further, it may be seen that the at least partial annulus defined by the one or more peripheral electrodes 14 may not necessarily be round or circular as shown in the exemplary embodiments illustrated in FIG. 1. For example, but without limitation, the at least partial annulus defined by the one or more peripheral electrodes 14 may also be elliptical, ovoid, polygonal, or combinations thereof. It is contemplated that in certain embodiments, the at least partial annulus may not be round, but may be polygonal, and in particular may be triangular, square, hexagonal, octagonal, or configured in other polygonal configurations.

The electrode geometry 12 may also comprise one or more interior electrodes 16 disposed interior to the at least partial annulus defined by the one or more peripheral electrodes 14. As may be seen by the various embodiments shown in FIG. 1, the interior electrode 16 may be configured in a number of configurations, including, for example but without limitation, circular (1A), hexagonal (1B), annular (1C), semi-annular (1D), or semicircular (1E). In addition to the pictured configurations, further configurations of the interior electrode 16 are contemplated, such as elliptical, semi-elliptical, ovoid, semi-ovoid, other polygonal configurations besides hexagonal such as triangular, square, or octagonal, star-shaped, or combinations thereof. The one or more interior electrodes 16 may comprise a single interior electrode 16, as in the illustrated embodiments of FIG. 1, but may also comprise embodiments in which the one or more interior electrodes 16 comprise multiple electrodes which together define a particular configuration.

The electrodes of the electrode geometry 12 may be formed of any material known to be useful in the formation of electrodes, including but not limited to copper or gold, or any other conductive metal or material or combination of metals or materials suitable for the particular purpose for which the electrode geometry 12 is intended. Further, it may be seen that the electrodes of the electrode geometry 12 may not necessarily be discrete portions from the surrounding substrate 18, but rather may be exposed regions of the substrate material itself which are not insulated via, for example, the presence of a lacquer or other masking material.

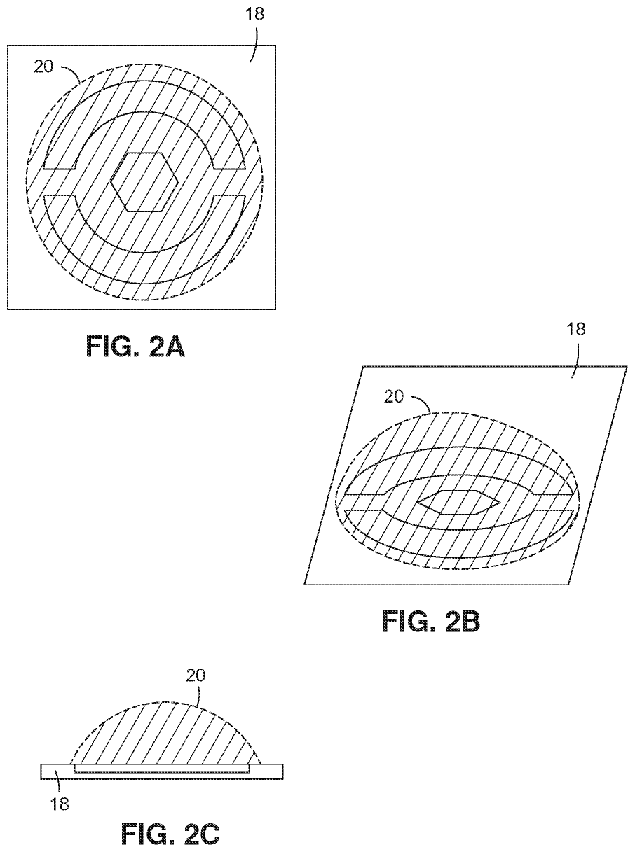

Turning now to FIG. 2, top (2A), perspective (2B), and side (2C) views of an exemplary embodiment of a system 10 for inducing toroidal mixing is shown. As may be seen, the system 10 for inducing toroidal mixing may comprise a substrate 18 in which the electrode geometry 12 may be disposed, and when in use, a volume of liquid 20 may be disposed in electrical contact with at least one of the one or more peripheral electrodes and at least one of the one or more interior electrodes.

The substrate 18 may comprise any material suitable for use as a substrate, and preferably is formed of a material being capable of being partially or completely electrically insulated so as to not interfere with the electrically current which may be applied to the electrodes of the electrode geometry 12. In the exemplary embodiment, the substrate 18 comprises a printed circuit board, and is formed of materials typically used to form printed circuit boards, including but not limited to phenol formaldehyde resin infused phenolic cotton paper, woven fiberglass cloth impregnated with an epoxy resin, an insulated metal substrate such as aluminum coated with a laminate material, a polyimide foil such as Kapton or UPILEX, or a polyimide-fluoropolymer composite foil, either of which may be utilized to form a flexible substrate, or other materials such as PTFE, alumina, or polyimide.

It may be preferred that the substrate 18 be substantially planar with the electrode geometry 12 likewise being substantially planar, or it may be preferred that the substrate 18 may be in a substantially nonplanar configuration, for example, a curved depression which may be preferred for retaining the volume of liquid 20. As such it may accordingly be seen that the electrode geometry may also be substantially nonplanar as well. It is additionally contemplated that the substrate 18 may be configured in a well 28 configuration wherein the electrode geometry may at least partially disposed within a well for at least partially containing the volume of liquid. Further, it may be preferred that the substrate 18 and the electrode geometry 12 be formed so as to present a substantially smooth surface, or it may be preferred that the electrode geometry 12 be formed to protrude above or below the level of the surface of the substrate 18. The electrode geometry 12 may also be advantageously defined by a solder mask.

The volume of liquid 20 may be any liquids or combinations of liquids suitable for mixing, including but not limited to solutions, suspensions, mixtures of solutions and/or suspensions, or aqueous, non-aqueous, polar, or nonpolar solutions or suspensions, or combinations thereof. In one exemplary embodiment, the volume of liquid 20 may be a combination of a solution containing erythrocytes from a first individual and plasma or serum from a second individual. However, it may be seen that the potential liquids suitable for mixing using the herein contemplated systems 10 for inducing toroidal mixing in a volume of liquid are essentially infinite, and are not to be limited merely to the herein disclosed exemplary embodiments. The volume of the volume of liquid 20 may be any volume that is desired to be mixed via toroidal mixing. Preferably, this volume is between 100 nL and 5 mL. More preferably, this volume is between 100 nL and 100 μL.

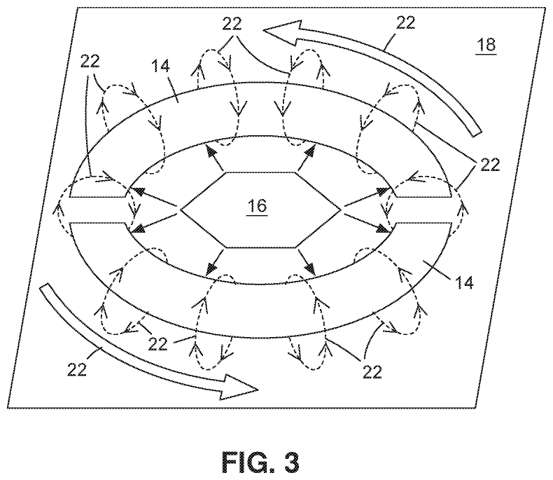

Turning now to FIG. 3, an illustration of the magnetic field lines that may resulting from application of a first electrical current to the one particular embodiment of the electrode geometry 12 of a presently contemplated system 10 for inducing toroidal mixing in a volume of liquid. As may be seen, when the first electrical current, which preferably is an alternating current, is applied to the exemplary electrode geometry 12, with a volume of liquid 20 (not pictured) in electrical contact with the electrode geometry 12, an induced electrical field may be induced in the volume of liquid 20 above the annular configuration defined by the peripheral electrodes 14. As a consequence of this induced electrical field, a toroidal mixing motion 22 may be induced within the volume of liquid 20. As may be seen by FIG. 3, this toroidal mixing motion 22 may be defined by, among other things, a horizontal mixing component planar to certain embodiments of the electrode geometry 12 wherein the volume of liquid 20 and its constituents may be mixed horizontally, as well as a vertical mixing component wherein the volume of liquid 20 and its constituents may be mixed vertically. This induced mixing motion 22 within the volume of liquid 20 may be the primary driver of the mixing action in the system 10 of the present disclosure. It may thus also be readily seen that the shape of the induced electrical field, and thus the shape of the induced mixing motion 22, may depend upon the particular configuration of the at least a partial annulus defined by the perimeter electrodes 14.

The particular parameters of the first electrical current that is to be applied to the electrode geometry 12 for inducing toroidal mixing in the volume of liquid 20 may be substantially variable depending upon a number of other considerations to be taken into account, including but not limited to the particular characteristics of the volume of liquid 20, such as its volume, conductance, polarity, polarizability, density, viscosity, the quantity and characteristics of any dissolved or suspended components therein, etc. Further, the parameters of the first electrical current that is to be applied to the electrode geometry 12 for inducing toroidal mixing in the volume of liquid 20 may also depend on the particular characteristics of the substrate 18 and the electrode geometry 12, such as size, shape, thickness, conductivity, resistance, etc. However, with routine experimentation, which may include modification of the characteristics of the waveform of the first electronic current, such as modifications in magnitude of the voltage peaks of any alternating current applied, the period of any periodic current applied, the form of the current (whether continuous, periodic, sinusoidal, sawtooth, square, etc.), whether a continuous or pulsed current is applied, and many other potential variations in the characteristics of the waveform. The only practical limitation on the application of the first electronic current to the volume of liquid 20 is that it is preferred that the application of the electronic current not induce electrolysis in the volume of liquid, which in water will generally commence at around 1.2 volts. However, it is generally contemplated that in most configurations, induction of toroidal mixing occurs when applying a symmetrical alternating current with a mean amplitude equal to zero and therefore electrolysis is avoided. However, a second applied waveform may include a DC offset that is exemplified by a waveform that has a non-zero mean amplitude. When said second waveform is applied to said volume of liquid electrolysis is avoided also by routine experimentation, which may include modification of the characteristics of the waveform of the first electronic current, such as modifications in magnitude of the voltage peaks of any alternating current applied, the period of any periodic current applied, the form of the current (whether continuous, periodic, sinusoidal, sawtooth, square, etc.), whether a continuous or pulsed current is applied, and many other potential variations in the characteristics of the waveform. The particular parameters of said electrical current that is to be applied to the electrode geometry 12 for inducing toroidal mixing concurrent with target aggregation at either said peripheral or center electrode in the volume of liquid 20 may be substantially variable depending upon a number of other considerations to be taken into account, including but not limited to the particular characteristics of the volume of liquid 20, such as its volume, conductance, polarity, polarizability, density, viscosity, the quantity and characteristics of any dissolved or suspended components therein, etc. Variables such as electrode composition may also affect the particular parameters of the electrical current to be applied. In exemplary embodiments, the first and second electronic currents may be generated by a signal generator. The signal generator may, in certain embodiments, be an external analog or digital arbitrary waveform generator, or may be a function performed on a digital logic controller or signal pattern generator, and as such, it is contemplated that the signal generator may be integral into the substance, such as in an integrated circuit.

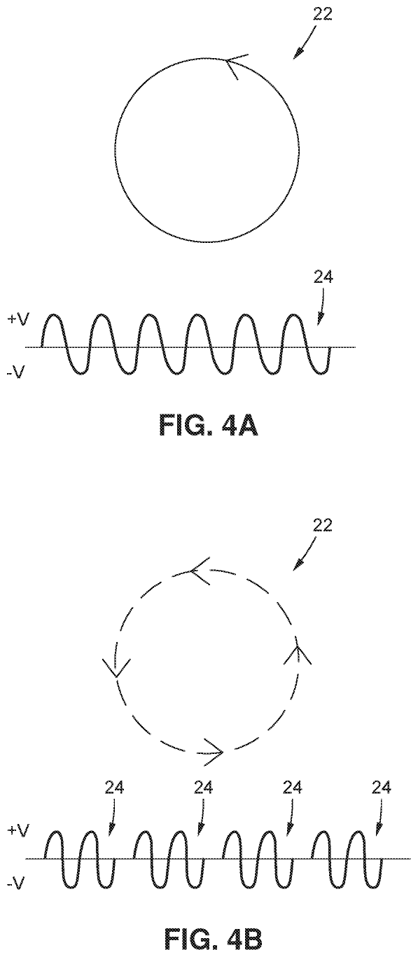

Turning now to FIG. 4, various embodiments of waveforms of the applied first electronic current are shown along with exemplary variations in the effect of the mixing motion 22 which may be expected to result from application of such waveforms. Illustratively, it may be seen by FIG. 4A that when a continuous sinusoidal waveform 24 is applied, it may be expected that a continuous mixing motion 22 may result in the volume of liquid 20. On the other hand, it may be seen by FIG. 4B that when pulses of a sinusoidal waveform 24 are applied, the mixing motion 22 which may be expected to result may be stepped, staggered, or substantially pulsed as well. It may thus be seen that depending on the desired characteristics of the mixing motion 22 to be induced, it may be desirable to use a continuous or a pulsed sinusoidal waveform. For example, in situations where a more aggressive and consistent mixing is preferred, a continuous waveform 24 may be correspondingly preferred. On the other hand, where a gentler mixing motion 22 may be desired, the delivery of a plurality of discrete impulses that may result from the use of a pulsed waveform 24 may be preferred. Likewise, it may also be seen that other variations in the waveform 24, such as periodicity, magnitude, etc. may also have a corresponding effect upon the mixing motion 22, and that such variation in effect of the mixing motion may be preferred or discouraged, depending on the application at hand.

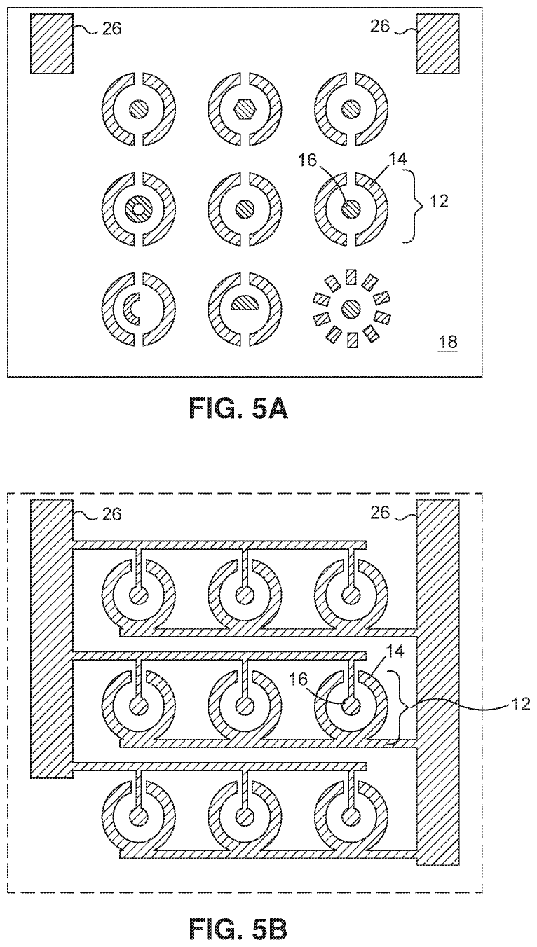

Turning now to FIG. 5, an illustration of an exemplary array of electrode geometries 12 formed in a printed circuit board is shown. FIG. 5A shows the surface of such an array, whereby the perimeter electrodes 14 and the interior electrodes 16 may be exposed and upon the surface of the substrate 18 whereupon they may electrically contact a volume of liquid 20. Further, contact points for power rails 26 are shown where an applied current may be delivered. FIG. 5B shows an interior layer of such an exemplary array, wherein each of the power rails 26 may be seen to extend to a respective group of either exterior electrodes 14 or interior electrodes 16. Thus, it may be seen that arrays of electrode geometries 12 according to the present disclosure may be readily fabricated via any known method of fabricated printed circuit board, such as lithographic or other methods.

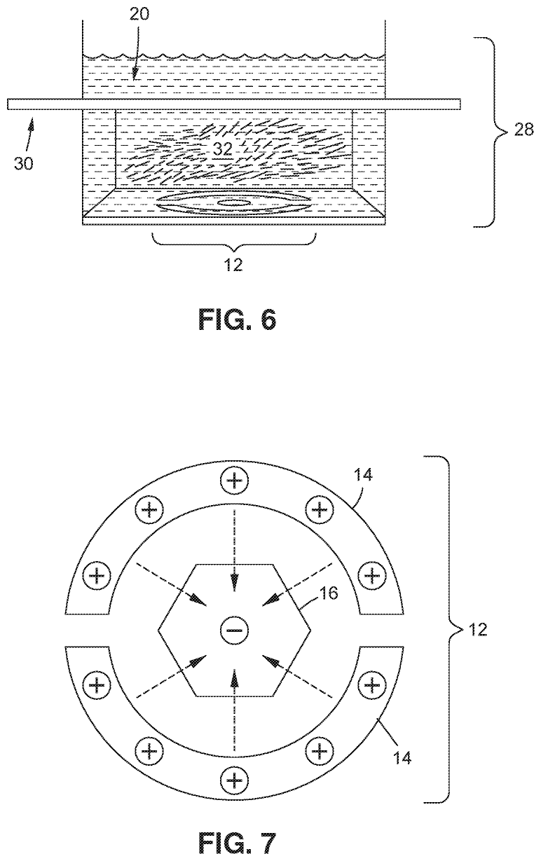

Turning now to FIG. 6, an illustration of one particular embodiment of a system 10 for inducing toroidal mixing in a volume of liquid 20 is shown, wherein the system 10 is configured as a well 28. According to this particular embodiment, the volume of liquid 20 is contained within the well with the electrode geometry 12 positioned at the base of the well. It may further be seen that a membrane 30 at least partially enclosing at least a portion of the well may be included within the system 10. According to various embodiments of the present disclosure, is contemplated that the membrane may be configured to be substantially permeable to at least one component of the volume of liquid 20 and substantially impermeable to a second component of the volume of liquid 20. Specifically in this particular embodiment, it is contemplated that the membrane 30 is substantially impermeable to a plurality of cells 32, while remaining substantially permeable to the remainder of the volume of liquid 20. In this way, it may be seen that the plurality of cells 32 may be confined within the well within the region defined by the membrane 30, while the remainder of the volume does not necessarily remain so confined. In this way, it may be seen that, for example, the plurality of cells 32 may be subjected to toroidal mixing at any time while other components of the volume of liquid 20 may be readily added or removed without requiring any particular process to prevent the plurality of cells 32 from being removed during performance of various sequential procedures upon the contents of the well 28. The membrane 30 may be, in certain embodiments, liquid films, frits, porous structures, or any other components or structures known to be at least partially permeable. Accordingly, it may be seen that via this particular embodiment of the system 10 for inducing toroidal mixing, sequential steps may be performed upon a volume of liquid with great speed, flexibility, and without the necessity for intrusion into the well for physical or other forms of agitation in order to accomplish mixing of the components of the volume of liquid 20 within the well 28.

Turning now to FIG. 7, it is illustrated how in certain embodiments, the electrode geometry 12 may be further operative to induce dielectrophoretic separation among components of the volume of liquid via application of a second electrical current to the electrode geometry 12. Specifically, it may be seen that such dielectrophoretic separation may be operative to cause at least one component of the volume of liquid to aggregate at a dielectrophoretic aggregation site. The dielectrophoretic aggregation site, in the illustrated embodiment, is the surface of the interior electrode 16. However, it may be seen that in other embodiments, the dielectrophoretic aggregation site may be elsewhere, including one or more other electrodes, or particular regions to which the dielectrophoretically separated component(s) may be induced to travel or be confined to. The second electrical current for inducing dielectrophoretic separation may be configured and optimized to the geometry of the system 10 according to known methods of configuring currents for dielectrophoretic separation, which may include, for example and without limitation, the second electrical current being one or more of: an alternating current, a direct current, or a variable current, and in altering other of the waveform characterizes of the second electrical current.

Accordingly, particular methods are contemplated in which the same volume of liquid 20 may be manipulated via both a mixing step and a dielectrophoretic separation, without requiring any application of physical contact upon or transport of the volume of liquid. In particular, and illustrative of the potential inherent in the system 10 herein described, it may be seen that a greatly superior method of performing a blood-type assay may be achievable via use of the system 10. In particular, a volume of liquid 20 comprising erythrocytes from a first individual and plasma or serum from another individual may be combined (preferably in situ at the electrode geometry 12) and positioned in electrical contact with at least one of the one or more peripheral electrodes 14 and at least one of the one or more interior electrodes 16, and thereupon toroidal mixing is induced via applying a first electrical current to the electrode geometry. Thereafter, the erythrocytes may be aggregated at an at a dielectrophoretic aggregation site by dielectrophoretic separation via applying a second electrical current to the electrode geometry. In the specifically contemplated embodiment, the dielectrophoretic aggregation site is the surface of the interior electrode 16, but in other embodiment, it may be other locations, such as the surface of the peripheral electrodes 14, a result that is easily achievable via simple reversal of the polarity of the second electronic current. Once the erythrocytes are aggregated at the dielectrophoretic aggregation site, the plasma or serum may be displaced from the dielectrophoretic aggregation site, for example, via application of a wash solution followed by siphoning or pipetting of the wash solution. It may thus be seen that the shape and configuration of the interior electrode, in this particular embodiment, may be an important aspect of performing this step, as the step of displacing plasma or serum while leaving the aggregated erythrocytes in place may be more difficult for certain configurations of the interior electrode. Subsequently, the dielectrophoretic aggregation site may be exposed to a solution comprising anti-human IgG, which may be seen to result in the agglutination of erythrocytes under certain blood type assay conditions, and may be seen to not result in agglutination of erythrocytes under other blood type assay conditions, and as such, one may be able to discern the blood type status of at least one of the first individual and/or the second individual as a consequence of interpreting such agglutination results. An extraordinary benefit of this method is that it allows adjustment of the aggressiveness of toroidal mixing to disrupt non-polymerized aggregates or pseudo-aggregates formed by weak non-specific interactions (e.g.: Van der Waals and Ion dipole), such as those found in non-polymerized erythrocytes (red blood cells or “RBC”) without disrupting true RBC aggregates caused by divalent binding of anti-human IgG and polymerization of RBC.

The above description is given by way of example, and not limitation. Given the above disclosure, one skilled in the art could devise variations that are within the scope and spirit of the disclosure herein. Further, the various features of the embodiments disclosed herein can be used alone, or in varying combinations with each other and are not intended to be limited to the specific combination described herein. Thus, the scope of the claims is not to be limited by the exemplary embodiments.

What is claimed is:

1. A system for inducing toroidal mixing in a volume of liquid, the system comprising:
a substrate; and
an electrode geometry at the surface of the substrate, the electrode geometry comprising one or more peripheral electrodes, the one or more peripheral electrodes together defining at least a partial annulus, and one or more interior electrodes, the one or more interior electrodes being disposed interior to the at least partial annulus defined by the one or more peripheral electrodes;
wherein when a volume of liquid is disposed in electrical contact with at least one of the one or more peripheral electrodes and at least one of the one or more interior electrodes, and when at least a first electrical current is applied to the electrode geometry, the system is operative to induce toroidal mixing in the volume of liquid.

2. The system of claim 1, wherein the at least partial annulus defined by the one or more peripheral electrodes is characterized as: circular, elliptical, ovoid, polygonal, or combinations thereof.

3. The system of claim 1, wherein the one or more interior electrodes are configured to be substantially concentric with the at least partial annulus defined by the one or more peripheral electrodes.

4. The system of claim 1, wherein the one or more interior electrodes are characterized as: circular, semicircular, elliptical, semi-elliptical, ovoid, semi-ovoid, annular, semi-annular, polygonal, star-shaped, or combinations thereof.

5. The system of claim 1, wherein the electrode geometry is at least partially disposed within a well for at least partially containing the volume of liquid.

6. The system of claim 5, wherein the system further comprises a membrane at least partially enclosing at least a portion of the well, the membrane being configured to be substantially permeable to at least one component of the volume of liquid and substantially impermeable to a second component of the volume of liquid.

7. The method of claim 6, wherein the volume of liquid comprises a plurality of cells, wherein the membrane is substantially impermeable to the plurality of cells.

8. The system of claim 1, wherein the system further comprises a signal generator for generating the first electrical current.

9. The system of claim 1, wherein the first electrical current is an alternating current and is characterized as: a continuous waveform, a pulsed waveform, a sinusoidal waveform, a non-sinusoidal waveform, or combinations thereof.

10. The system of claim 1, wherein the substrate comprises a printed circuit board.

11. The system of claim 1, wherein the system comprises an array having a plurality of electrode geometries at the surface of one or more substrates.

12. The system of claim 1, wherein the system is further operative to induce dielectrophoretic separation among components of the volume of liquid via application of a second electrical current to the electrode geometry.

13. The system of claim 12, wherein the dielectrophoretic separation is operative to cause at least one component of the volume of liquid to aggregate at a dielectrophoretic aggregation site.

14. The system of claim 12, wherein the second electrical current is one or more of: an alternating current, a direct current, a variable current.

15. A method of inducing toroidal mixing in a volume of liquid, the method comprising the steps of:
providing an electrode geometry at the surface of a substrate, the electrode geometry comprising one or more peripheral electrodes, the one or more peripheral electrodes together defining at least a partial annulus, and one or more interior electrodes, the one or more interior electrodes being disposed interior to the at least partial annulus defined by the one or more peripheral electrodes;
disposing the volume of liquid in electrical contact with at least one of the one or more peripheral electrodes and at least one of the one or more interior electrodes; and
applying a first electrical current to the electrode geometry, the first electrical current being an alternating current.

16. The method of claim 15, wherein the volume of liquid is between 100 nL and 5 mL.

17. A method of performing a blood-type assay, the method comprising the steps providing an electrode geometry at the surface of a substrate, the electrode geometry comprising one or more peripheral electrodes, the one or more peripheral electrodes together defining at least a partial annulus, and one or more interior electrodes, the one or more interior electrodes being disposed interior to the at least partial annulus defined by the one or more peripheral electrodes;
disposing a volume of liquid comprising erythrocytes from a first individual and plasma or serum from another individual in electrical contact with at least one of the one or more peripheral electrodes and at least one of the one or more interior electrodes;
inducing toroidal mixing in the volume of liquid via applying a first electrical current to the electrode geometry, the first electrical current being an alternating current;
aggregating the erythrocytes at a dielectrophoretic aggregation site by dielectrophoretic separation via applying a second electrical current to the electrode geometry;
displacing the plasma or serum from the dielectrophoretic aggregation site; and
exposing the dielectrophoretic aggregation site to a solution comprising anti-human IgG.

18. The method of claim 17, wherein the dielectrophoretic aggregation site is chosen from: at least one of the one or more peripheral electrodes, at least one of the one or more interior electrodes, another electrode, a region of an electric field, or combinations thereof.

19. The method of claim 17, wherein the step of displacing the plasma or serum is performed via application of a wash solution.

20. The method of claim 17, further comprising a step of detecting the relative presence or relative absence of cross-linked erythrocytes.
